(19) United States
(12) Patent Application Publication
(10) Pub. No.: US 2021/0017400 A1
(43) Pub. Date: Jan. 21, 2021

(54) Molecularly Well-defined Antibiofouling and Polyionic Coatings

(71) Applicant: ACatechol, Inc., Santa Barbara, CA (US)

(72) Inventor: Roscoe T. Linstadt, Palo Alto, CA (US)

(73) Assignee: ACatechol, Inc., Santa Barbara, CA (US)

(21) Appl. No.: 16/932,332

(22) Filed: Jul. 17, 2020

Related U.S. Application Data
(60) Provisional application No. 62/876,404, filed on Jul. 19, 2019.

(51) Int. Cl. C09D 5/14; C09D 5/16; C08L 83/04

(52) U.S. Cl. CPC C09D 5/14; C09D 5/1637; C08L 2203/16; C08L 83/04; C08L 2203/02; C09D 5/1662

(57) ABSTRACT

The present application discloses molecularly well-defined antibiofouling and polyionic coatings, materials and methods of use.

### CROSS-REFERENCE TO RELATED APPLICATIONS

This application claims the priority under 35 USC 119(e) to provisional application No. 62/876,404, filed Jul. 19, 2019.

### TECHNICAL FIELD

The present application relates to antibiofouling and coatings.

### BACKGROUND

In the United States alone, each year it is estimated that over 250,000 bloodstream infections are incurred in patients with silicone catheters, primarily due to microbial colonization of such indwelling devices. (Wallace, 2017) Bloodstream infections are frequently fatal, and severe cases result in hospitalization with costs up to 30,000 USD per infection incurred (Mermel, 2000). Colonization of surfaces by pathogenic bacteria is contingent on the prior formation of biofilms, which are complex matrices of exopolysacchharides, biomolecules and biopolymers. Once the biofilms are formed, they then serve to anchor sessile communities of microorganisms (Donlan 2002). Although antibiotic treatment can successfully kill off the adhered microbes, it does nothing to remove the remaining biofilm, and the fouled surfaces are highly susceptible to repeated colonization which increases the chances of re-infection. Current clinical practice attempts circumvent this problem by frequent regular replacement of the silicone, a concession to the fact that the materials employed presently do not provide protection against microbial colonization, and as such, materials or surface coatings that eliminate and provide long term resistance to biofilm formation are urgently required.

Most strategies that target initial biofilm formation involve generation of a surface with energetically favorable hydrophilic interactions at the interfacial boundary, usually by the incorporation of charged moieties such as quaternary ammonium salts or neutral, polar moieties such as PEG (Wallace 2017). Recent reports detailing antibiofouling catheter surfaces involve polymeric materials functionalized with both charged and neutral hydrophilic residues, yet these are incorporated by means of their parent (meth)acrylate or cyclic carbonate monomer (Ding 2012, Smith 2012, Vaterrodt 2016). The use of acrylic and cyclic carbonate monomers contributes to the overall percentage of hydrocarbons in the coating, a disadvantage as this parameter is known to increase susceptibility to biofilm formation. Although there is precedence for using silanization technology to immobilize zwitterions upon a surface, it is limited to a single silanization reagent which does not reliably give uniform monolayers, nor does it allow for substantial variations in the ionic coating structure, and achieves only about ⅔ conversion of active surface sites to zwitterionic carboxy-betaine functionality, a consequence of the silanization reagent employed (Huang, 2014). It is well established that zwitterionic, dicationic and gemini surfactants are surface active at concentrations orders of magnitude lower than traditional singly ionic surfactants (Menger, 2000). The surface activity in all cases is greatly enhanced by the addition of a second ion, and greatest still in the latter case in which there is forced separation of two or more hydrophobic chains by multiple charged moieties. Even still, most biocidal antifouling coatings make use of less active PEG residues, or only singly ionic quaternary ammonium residues. Zwitterions when employed, are often terminated at the anionic site (as in carboxy- and sulfono-betaines) despite the fact that anionic residues at the interfacial boundary are not as effective as biocidal agents as cationic residues. The higher surface activity of gemini surfactants notwithstanding, to the present inventor's knowledge, no attempts have been made to translate their structural principles into antifouling and anticorrosion materials.

### SUMMARY OF THE INVENTION

It is desirable to prepare a class of durable and molecularly well defined antibiofouling polyionic coatings for biomedical devices or other surfaces where biofouling is problematic. In one embodiment, the present application discloses methods by which biomedical surfaces may be transformed into reactive monolayers, and then coupled with polyionic reagents to arrive at the well defined antibiofouling coatings. The present application describes unique classes of polyionic coupling reagents used to impart surfaces with antibiofouling properties. The present application also discloses methods of translating the properties of gemini surfactants into antibiofouling surface coatings.

### Definitions

The following definitions given are provided for clarification purposes only, and are only generally indicative of the concepts so described. The following list of definitions is not to be regarded as all-inclusive with regard to the concepts that must be grasped to understand the present invention, and a reader may have to refer to the primary literature referenced herein if a concept or term is unfamiliar. A person skilled in the relevant art will grasp that different definitions other than the ones given in this specification may be employed without substantially changing the essential meaning, overall intent, and broad concepts of the present invention.

“Biofilm” refers to the mixtures of biomolecules, biopolymers, exopolysachharides, and other materials that serve to anchor microorganisms to various surfaces. “Biofilm” may be used to refer to such mixtures both with and without adherent microorganisms.

“Antibiofouling” and “antifouling” refer to the property of something, such as a material, in which it discourages the formation of biofilms and/or the adherence of microorganisms.

“Gemini surfactant” generally refers to a surfactant with two or more polar head-groups separated by a spacer and two or more hydrophobic tails. For a detailed definition and descriptions of the various types of Gemini-Surfactants refer to: Menger, F. M. et al. “Gemini Surfactants” Angew. Chem. Int. Ed. 2000, 39, 1906-1920.

“Zwitterionic salts” refer to molecules that possess both zwitterionic functionality and ionic salt functionality. As such zwitterionic salts have a minimum of 4 charges. For a more detailed definition and description, refer to: Blesic, M. et al. “An Introduction to Zwitterionic Salts” Green Chem., 2017, 19, 4007-4011.

“SAM” is an abbreviation for “Self-assembled-monolayer” and is well known and defined in the art.

“Reactive monolayer” refers to a molecularly thin layer that possesses reactive functionality, capable of forming covalent bonds with suitable coupling partners; and as disclosed herein.

“Silicone” refers to compounds and polymers comprised of chains of alternating silicon atoms and oxygen atoms.

“PDMS” refers to poly-dimethylsiloxane.

“PET refers to polyethylene terephthalate, commonly abbreviated PET, PETE, (or the obsolete PETP or PET-P).

“PVC” refers to polyvinyl chloride.

PU refers to polyurethane.

PMMA refers to polymethyl methacrylates.

“Polyionic coupling agent” refers to any molecule possessing two or more permanent charges, that is capable of forming covalent bonds with other molecules based upon the functionality contained within. For example, a molecule possessing two-quaternary ammonium residues, and a carboxylic acid may be considered a polyionic coupling agent as it possesses two charged sites, and may be coupled with amines to form amide bonds by the appropriate protocol of activation of the carboxylic acid, prior to coupling.

When images are used in the present disclosure to describe covalent surface linkages from an organosilicon compound to a surface such as:

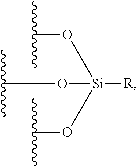

it is to be understood that not all of the oxygen atoms are necessarily directly attached to the surface, as alkoxyorganosilanes may undergo various degrees of hydrolysis and oligomerization polymerization with water in a silanizing mixture before condensing upon to surface hydroxyls. This phenomenon is described in: Naik, V.; Crobu, M.; Venkataraman, N. V.; Spencer, N. D. “Multiple Transmission-Reflection IR Spectroscopy Shows that Surface Hydroxyls Play Only a Minor Role in Alkylsilane Monolayer Formation on Silica” J. Phys. Chem. Lett. 2013, 4, 2745-2751. Thus, such images depicting oxygen attachment are used to describe organosilicon surface coatings that are covalently attached to the surface, but may be partially oligomerized to contain multiple Si—O—Si linkages in between Surface-O—Si linkages.

Likewise, when the phrase that is used such as “the silyloxy portion(s) depicted above (—(O)₃Si—) is covalently bonded to a surface”, it is to be understood that not all of the oxygen atoms are necessarily directly attached to the surface, and may be partially oligomerized.

In both instances, both pictorially and via text, the intention and spirit of the nomenclature used is to indicate the covalent nature of the coating, which is distinct from silanes which have been purposefully polymerized with water to obtain silicone polymers such as trihydroxysilanes, polysiloxanes, and sesquisiloxane polymers.

### BRIEF DESCRIPTION OF THE FIGURES

FIG. 1 is a representative ¹H nmr spectrum of the compound that is N¹,N¹,N³,N³-tetramethyl-N¹,N³-bis(3-(trimethoxysilyl)propyl)propane-1,3-diaminium iodide prepared from Example 1.

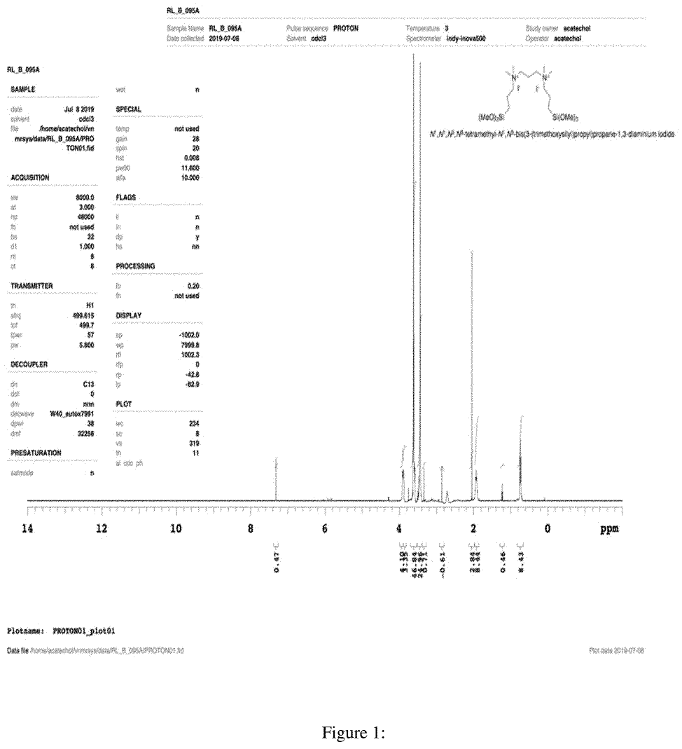

FIG. 2 is a representative of a ¹³C nmr spectrum of the compound that is N¹,N¹,N³,N³-tetramethyl-N¹,N³-bis(3-(trimethoxysilyl)propyl)propane-1,3-diaminium iodide prepared from Example 1.

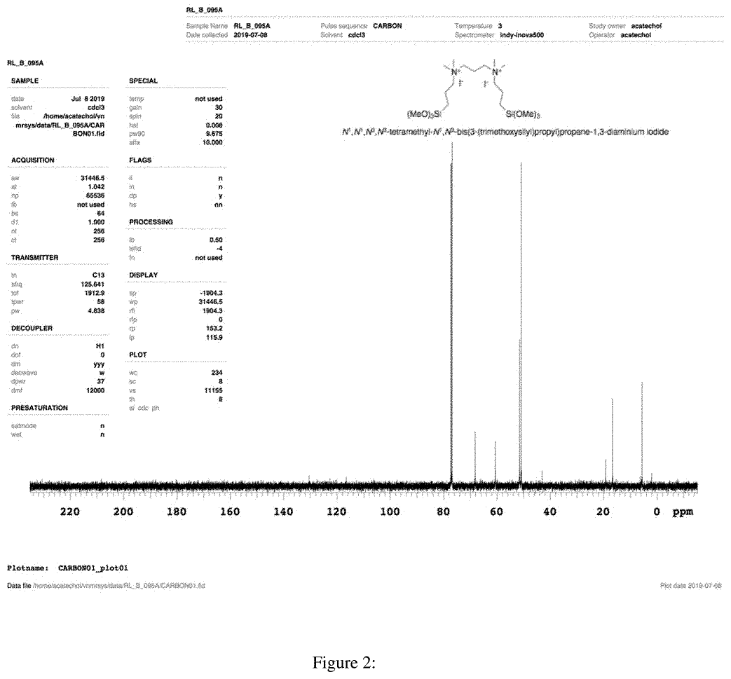

FIG. 3 is a representative ¹H nmr spectrum of the compound that is hex-5-en-1-yl (2-(trimethylammonio)ethyl) phosphate prepared from Example 5.

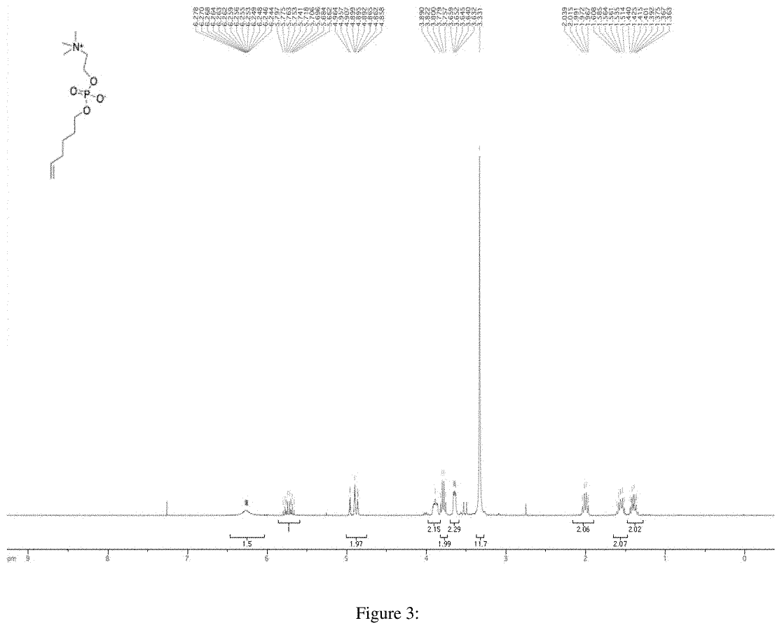

FIG. 4 is a representative ¹H nmr spectrum of the compound that is N¹,N¹,N¹,N²,N²-pentamethyl-N²-(pent-4-en-1-yl)ethane-1,2-diaminium bromide iodide prepared from Example 7.

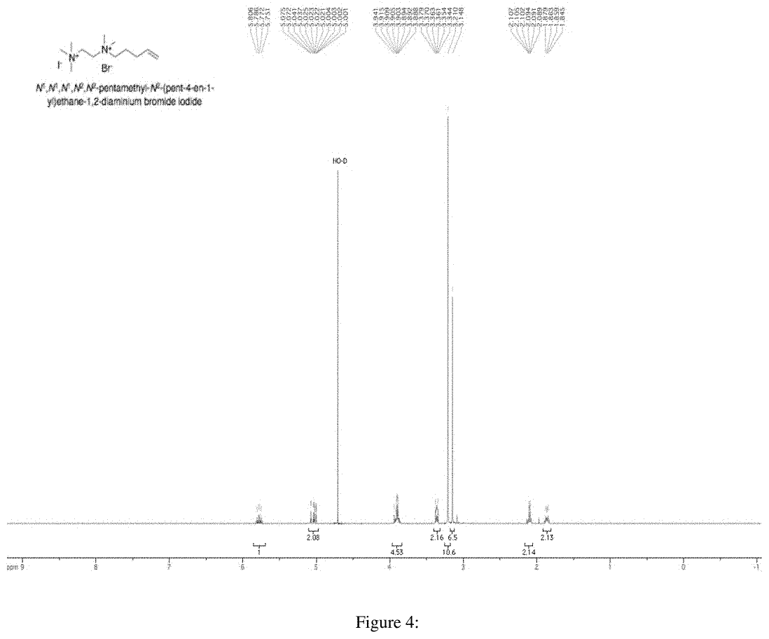

FIG. 5 is a representative ¹H nmr spectrum of the compound that is N¹,N¹,N²,N²-tetramethyl-N¹,N²-di(pent-4-en-1-yl)ethane-1,2-diaminium bromide prepared from Example 8.

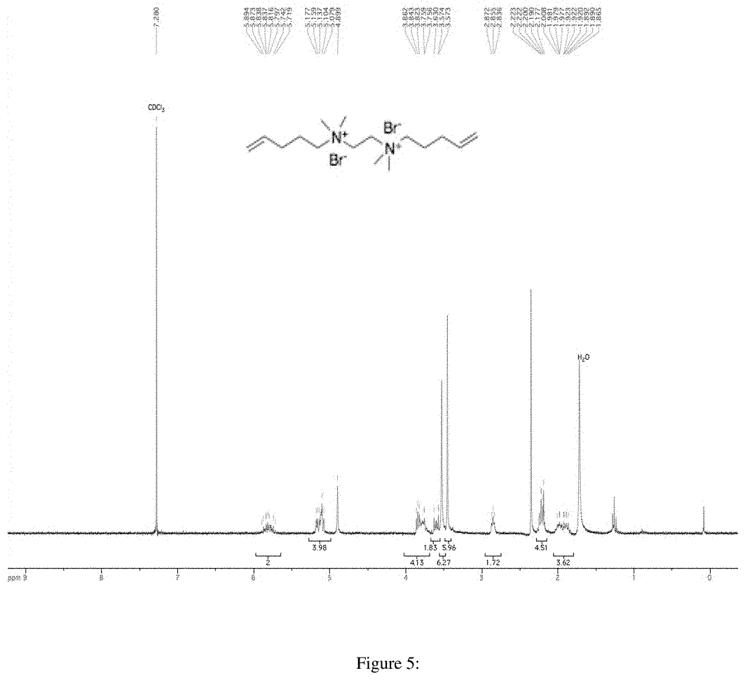

FIG. 6 is a representative depiction of the water droplet contact angle measurements of a representative library of gemini-surfactant inspired polyionic surface coatings.

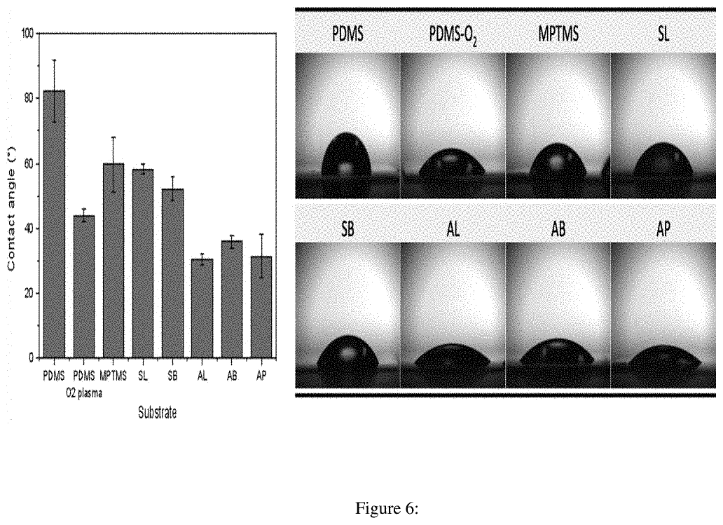

FIG. 7 is a representative depiction of unmodified silicone surfaces shown with fully colonized (left) while a gemini-AB-coated surface (right), and shows almost no living bacteria attached to the surface.

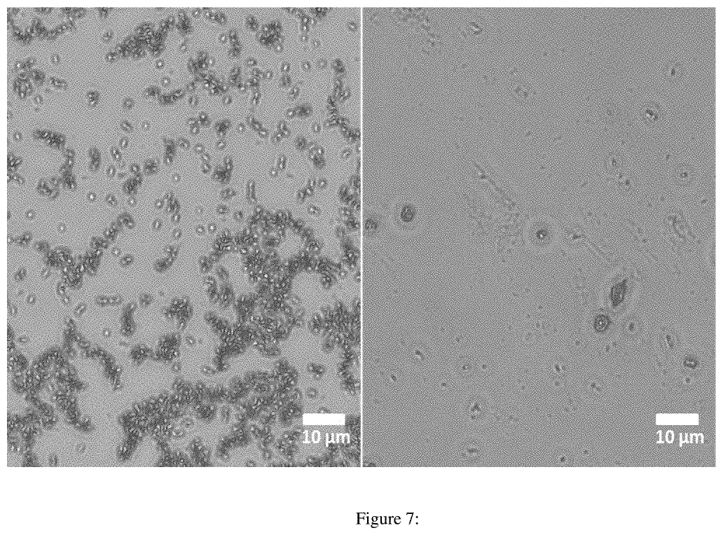

### DETAILED DESCRIPTION OF THE EMBODIMENTS

The present invention may be embodied in other specific forms without departing from its spirit or essential character. The described embodiments are to be considered in as illustrative and not restrictive. A skilled practitioner in the art will recognize that other similar or equivalent components and methods not explicitly delineated herein may be utilized without departing from the broad and general concepts of the present invention. All changes which come within the meaning and range of equivalency of the claims are to be embraced within their scope. All references cited anywhere within this present specification are incorporated by reference into this disclosure.

In some embodiments the present application describes a novel class of ionic coatings that may be generated or applied to various surfaces, to render the coatings resistant to biofouling processes. In some aspects the coatings are structurally similar to gemini surfactants, but differ mainly by being immobilized upon a surface. Such coatings may be termed “gemini-inspired-surfaces” or “gemini-inspired-coatings”. In some aspects the coatings differ from extant biofouling coatings in that they are molecularly thick and are not based upon polymers. In some embodiments the precursors to the coating may themselves be gemini surfactants or other polyionic detergents. In some embodiments the anions of the ionic coatings are halides. In other embodiments the coatings may be carboxylates, phosphates, phosphonates, sulfates, sulfonates or sulfinates. In other embodiments, the anions may be weakly coordinating such as triflates, triflimides, tetrafluoroborate, hexafluorophosphate and hexafluoroantimonate. By careful selection of the ions in the salt, the solubility profiles may be altered. In other embodiments the anions may be other biochemicals or agents such as oxalate, pyrophosphate and tartrate. In some embodiments the ionic coating may possess a mix of different anions. Additionally, by placing the coatings and compounds of the present application into different salt mixtures, salt metathesis and exchange may occur giving rise to compounds and coatings with different counterions not explicitly delineated herein.

In some embodiments these gemini-inspired surface coatings possesses interfacial physical properties dictated by the organosilane employed, and may be adjusted accordingly by modifications to the silane's molecular structure. Despite their superiority to conventional monoionic surfactants, gemini-surfactants have not yet been translated into virucidal/antimicrobial coatings. By incorporating their key structural aspects into immobile silane coatings, hydrophilicity may be significantly improved, limiting the ability of infectious pathogens to settle upon the surfaces. The covalent attachment of the silane increases durability and prevents adventitious moisture from dissolving away the nominally water-soluble ionic residues.

In some embodiments the present application describes coatings covalently linked to a surface via a covalent Si—O linkages, where the organic portion of the molecule contains multiple permanent charges. In some embodiments the present application describes a method of coating a surface via silanization reactions, and subsequently coupling it to another molecule containing multiple ionic residues to arrive at charged surface coatings. In other embodiments the present application describes a class of ionic silanization reagents that may be used to directly create a well defined ionic antibiofouling surface. In some embodiments the surface of interest is first coated with a thiol functionalized trialkoxysilane, such as mercaptopropyltrimethoxysilane (commonly abbreviated MPTMS or MPS) to achieve a thiol functionalized self assembled monolayer upon the surface. The functionalized monolayer may then be reacted with an ionic compound. For representative procedures detailing the use of thiol-ene reactions between a thiol functionalized silane and a hydrophilic alkene, see “Thiol-ene Click Reaction as a General Route to Functional Trialkoxysilanes for Surface Coating Applications” Tucker-Schwartz et. al. J. Am. Chem. Soc., 2011, 133, 29, 11026-11029.

Surface hydroxylation methods: In some embodiments surfaces are hydroxylated prior to engaging in silanization reactions. If a surface does not possess sufficient hydroxyl moieties it may necessitate hydroxylation prior to engaging in silanization. For example, PDMS surfaces which are normally inert to silanization reactions may be hydroxylated by immersion in aqueous solutions containing acids such as H₂SO₄ or HCl and hydrogen peroxide or other oxidizing agents. For example, a PDMS surface may be submerged in a “pirhana” type solution containing 50% concentrated sulfuric acid and 50% of (30% wt/v) H₂O₂. In one aspect solutions may contain variable amounts of sulfuric acid, and variable amounts hydrogen peroxide. Such solutions comprised of variable mixtures of hydrogen peroxide and sulfuric acid by producing the unstable oxidizing acid persulfonic acid in situ, which then achieves hydroxylation of the surface. In other embodiments, other oxidizing agents, and oxidizing acids may be used, whether applied as a directly or generated in situ analogously to the pirhana solutions. In other embodiments the mixture used to hydroxylate the surface is a solution composed of an acid such as HCl, HBr, HI, H₃PO₄, AcOH, trifluoroacetic acid, perfluorooctane-sulfonic acid, trifluoromethanesulfonic acid, chlorosulfonic acid, optionally mixed with variable amounts of aqueous hydrogen peroxide. In one embodiment, the surfaces may be cleaned with various strengths of aqua regia, which are mixtures of HCl and HNO₃. In other embodiments, nitric acid may be used alone to hydroxylate the surface. In some embodiments pure white fuming nitric acid is used, where in other embodiments red fuming nitric acid is used. In other embodiments, nitric acid is used as an aqueous solution. In other embodiments, “mixed acids” may be used to hydroxylate the surface such as nitro-sulfuric acid, nitric/acetic acid mixtures. In other embodiments the oxidizing acids are applied directly to the surface to achieve hydroxylation, instead of being generated in situ. For example, surface hydroxylation may also be achieved by application of peracetic acid. In other embodiments, surface hydroxylation may be achieved with other of oxidizing agents such as O₂, Cl₂, Br₂, I(OAc)₃, F₂, I—Cl, BrF₃, BrCl₃, Ozone, PhI(OAc)₂, either alone, in the vapor phase, or as composite solutions in water, or suitably inert solvents. In some variations, ultraviolet light is used in combination with those methods described above to assist in surface activation.

In some embodiments, the surfaces are oxidized and/or hydroxylated by application of plasma cleaning techniques using air plasma, oxygen plasma or nitrogen plasma. For representative procedures and examples of plasma oxidation that may be used in the present application, see: “Oxygen plasma treatment for reducing hydrophobicity of a sealed polydimethylsiloxane microchannel” Tan et. al., Biomicrofluidics, 2010, 4, 032204; “Hydrophilic surface modification of PDMS for droplet microfluidics using a simple, quick, and robust method via PVA deposition” Trantidou et. al., Microsystems & Nanoengineering, 2017, 3, 16091.

In some embodiments, after surface hydroxylation, the surface is washed thoroughly with water, alcohols, or other solvent before being thoroughly dried. In general, it is usually necessary to remove adsorbed surface moisture prior to silanization as these can react competitively with the surfaces with the trialkoxysilyl group. In general, the lower the amount of surface moisture, and higher purity of the silanization agent, the higher degree of uniformity to the self assembled monolayer. In other embodiments, after oxidizing the surfaces by plasma cleaning, the surface may be immediately reacted with the silanization reagent without an added rinsing step.

Silanization methods. In some embodiments, surfaces containing hydroxyl functionalities are reacted with a silanization reagent, such as a trialkoxysilane, in an inert or otherwise anhydrous solvent for a period of time to achieve formation of Si—O bonds to the surface. For example, a surface hydroxylated by any of the aforementioned methods, after drying under inert gas, may be silanized by submerging it in a 5% wt/v solution of mercaptopropyl trimethoxysilane in anhydrous toluene for 30 minutes. The concentration of the silanization reagent, choice of silanization reagent, reaction time, water percentage, choice of surface choice of solvent can all be varied. After the desired amount of time has elapsed, the surface may be removed from the silanization solution, rinsed with additional solvent to remove unreacted monomer, and then dried, with optional further curing. In some variations, the water content of the solvent used in the silanization step may be 0-0.00001% (v/v), 0.00001-0.0001% (v/v), 0.0001-0.001% (v/v), 0.001-0.01% (v/v), 0.01-0.1% (v/v), 0.1-1% (v/v), or 1-10% (v/v). In one aspect the water content of the solvent used in the silanization process affects the morphology, uniformity, thickness, and density, of the layer deposited on the surface. In another related aspect, the water content affects the time required for complete silanization of the surface.

The following methods, procedures, and associated reagents, in addition to those described or cited elsewhere in this disclosure, may be used to prepare the silanized coatings of the present application, which are described in references such as: Plueddemann, E. P. Silane coupling agents, 2nd ed.; Plenum Press: New York, 1991; Chapter 2; Krasnoslobodtsev, A. V.; Smirnov, S. N. Langmuir 2002, 18, 3181-3184; Brentel et. al. Dental Materials, 2007, 23, 1323-1331; McGovern, M. E.; Kallury, K. M. R.; Thompson, M. Langmuir, 1994, 10, 3607-3614; Cras et. al. Biosensors & Bioelectronics, 1999, 14, 683-688.

In some embodiments, silicon, PDMS, PET, PETG, PC or PVC is grafted with alkyl- or perfluoroalkyl silanes after preliminary oxygen/nitrogen plasma treatment. Surface modification of the polymer is performed by oxygen and/or nitrogen plasma treatment based on Surf. Interface Anal., 40: 1444-1453. The gas pressure is fixed at 75 Pa and the discharge power was set to 200 W. Surface wettability is determined by water contact angle measurements. The contact angle of a water droplet decreases from 75 for the untreated sample to approx. 200 for oxygen-, and approx. 25 for nitrogen-plasma treated samples for 3 seconds. Contact angles decrease with extended treatment time for both plasmas and reach about 100 for nitrogen plasma and <100 for oxygen plasma after 1 min of plasma treatment. The highly oxidized polymer surface is then grafted with organosilanes.

For example, self assembled monolayers (SAM) may be formed upon plasma treated surfaces of the present invention by the following a silanization method adapted from: Naik, V. V.; Crobu, M.; Venkataraman, N. V.; Spencer, N. D. “Multiple Transmission-Reflection IR Spectroscopy Shows that Surface Hydroxyls Play Only a Minor Role in Alkylsilane Monolayer Formation on Silica” J. Phys. Chem. Lett. 2013, 4, 2745-2751. In a representative procedure, a dilute (0.1 mM) solution of octadecyl trichlorosilane (abbreviated OTS) is prepared in freshly distilled decahydronaphthalene (decalin) (cis-trans mixture) (Sigma Aldrich) to coat a 30×18 mm² section of PET, PETG, PVC, PU or silicone surface, that had been plasma treated as described earlier. The plasma treated surface is then immersed in the OTS solution for 30 minutes at ambient temperature to obtain the OTS-coated surface. The surface is then cleaned by sonicating in toluene and then dried under a stream of dry nitrogen. The formation of a SAM may be confirmed by variable-angle spectroscopic ellipsometry (VASE) (M-2000FTM, J. A. Wollam Inc., Lincoln, USA) and static-contact-angle measurements (Model 100, Ramd Hart Inc., USA). The OTS film thickness is measured as the difference in the optical thickness of a blank silicon wafer and its thickness after coating with OTS. The data is evaluated using the WVASE32 software (WexTech Systems, Inc., New York, USA). For water and hexadecane-contact-angle measurements, 3 μl of solvent is used.

In some embodiments, the functionalized surface may then be subsequently coupled with the appropriate polyionic coupling agents. For example, the above mentioned mercapto-functionalized surface may then be coupled with a molecule possessing two quaternary ammonium salts, and a terminal olefin by submerging the mercapto functionalized surface in an aqueous solution containing the coupling agent, then irradiated with ultraviolet light for a period of time, before removing the surface from the solution, rinsing and drying to achieve a polyionic molecularly well defined antibiofouling surface.

In some embodiments, the surface that is functionalized is a metal, metal oxide, mineral, or mineral oxide that is part of a biomedical device. In some embodiments, the biomedical device may be a dental appliance.

In some embodiments the coatings may be used in oropharyngeal feeding tubes, urinary catheters, central venous catheters, hemodialysis catheters, peritoneal dialysis catheters and other indwelling medical devices where biofouling/healthcare-acquired infections are problematic.

In some embodiments the surfaces functionalized are those commonly found in the dental field, including, but not limited to: teeth, hydroxyapatite, dental resins, brackets, crowns and braces. In other embodiments the surfaces functionalized are those commonly found in cosmetics such as fingernails, toenails, skin, acrylic dyes and jewelry. In other embodiments, the surfaces that may be functionalized are wood, paint, cloth, cellulose, metal, metal-oxides, ceramics, clays, glass, rubbers or plastics.

In some embodiments, the surfaces functionalized are those commonly found in indwelling biomedical devices such as catheters and endotracheal tubes, such as silicone, PDMS, PVC, PET, PETG, PU and PC.

Silatranes are a class of trialkoxy-silicon compounds with a tripodal ligand on silicon such as triethanolamine, that possesses a transannular dative N—Si bond rendering the silicon atom formally pentavalent. Both the tripodal ligand and dative N—Si bond render organosilatranes substantially more stable and resistant to moisture than conventional organotrialkoxysilanes. In alcoholic solutions, organosilatranes derived from triethanolamine may be converted to organotrialkoxysilanes by addition of acid such as acetic acid, which protonates the atrane nitrogen and catalyzes the removal of triethanolamine and exchange with the alcoholic solvent.

In some embodiments, trialkoxysilane-polyionic silanization reagents may be generated in situ from organo-silatranes and triethanolamine silantranes, such as N(CH₂CH₂O)₃—SiR. Silatrane analogs of the polyionic coupling agents may then generate the polyionic coupling agents in-situ, by addition of acids to exchange the triethanolamine for alkoxy ligands and can arrive at the polyionic surface coatings and polyionic silanization reagents described elsewhere in this disclosure.

In some embodiments, analogous silatranes are used as moisture-stable precursors to trialkoxyorganosilane polyionic surface modifying agents of the present invention. In some aspects, this allows facile isolation and characterization of polyionic silanes that would be otherwise possessed of low shelf life as trialkoxy-polyionic silanization reagents are both hygroscopic and moisture reactive.

The following procedures may be employed for the preparation of the compounds of the present application. The starting materials and reagents used in preparing these compounds are either available from commercial suppliers such as the Aldrich Chemical Company (Milwaukee, Wis.), Bachem (Torrance, Calif.), Sigma (St. Louis, Mo.), or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989. Organic Syntheses, Collective, vols 1-12, John Wiley and Sons, New York, N.Y. In some cases, protective groups may be introduced and finally removed. Suitable protective groups for amino, hydroxy and carboxyl groups are described in Greene et al., Protective Groups in Organic Synthesis, Second Edition, John Wiley and Sons, New York, 1991. Standard organic chemical reactions can be achieved by using a number of different reagents, for examples, as described in Larock: Comprehensive Organic Transformations, VCH Publishers, New York, 1989.

In one embodiment, the present application describes polyionic surface coatings of the formula I:

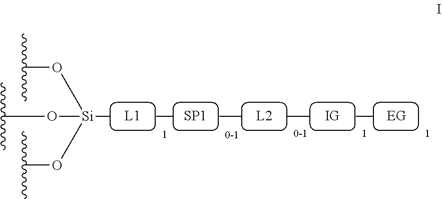

wherein:
the silyloxy portion (i.e., —(O)₃Si—) is covalently bonded to a surface.
L1 is a methylene spacer between 2 and 10 carbons in length e.g. —[(CH₂)₂₋₁₀]—
each SP1 is optionally a spacer selected from

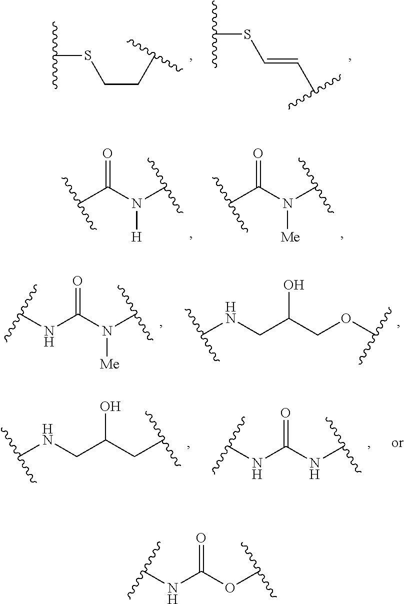

with optionally 0 or 1 spacers L2, where L2 is a methylene spacer between 1 and 8 carbons in length e.g. —[(CH₂)₁₋₈]—;
G is a polyionic group selected from the following:

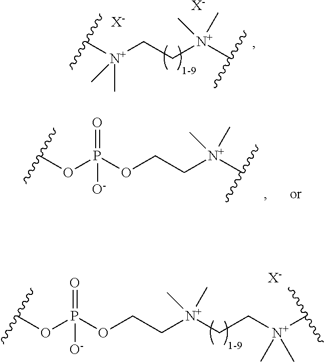

EG is the end group selected from:
   methyl, —[(CH₂CH₂O)₁₋₃₀]-Me, —[(CH₂CH₂O)₁₋₃₀]—H, or a linear n-alkyl chain between 2 and 8 carbons in length, or between 2 and 20 carbons in length; and each X− is independently an anion selected from Cl⁻, Br⁻, I⁻, F⁻, SO₄²⁻, PO₄³⁻, CO₃²⁻, CH₃SO₃⁻, CF₃SO₃⁻, BF₄⁻, TsO⁻, AcO⁻, BzO⁻ and NTf₂⁻.

In another embodiment the present application describes a class of polyionic surface coatings of the formula II:

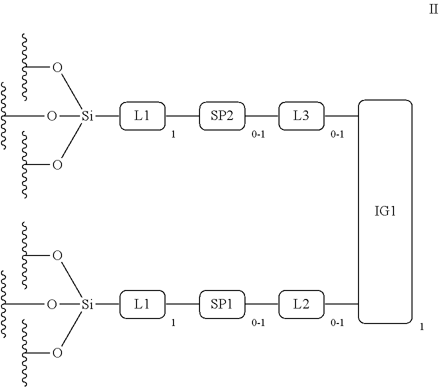

wherein:
the silyloxy portions depicted above is covalently bonded to a surface;
each L1, L2 and L3, where present, is independently a methylene spacer between 2 and 10 carbons in length;
wherein each SP1 and SP2, where present, is a spacer selected independently from

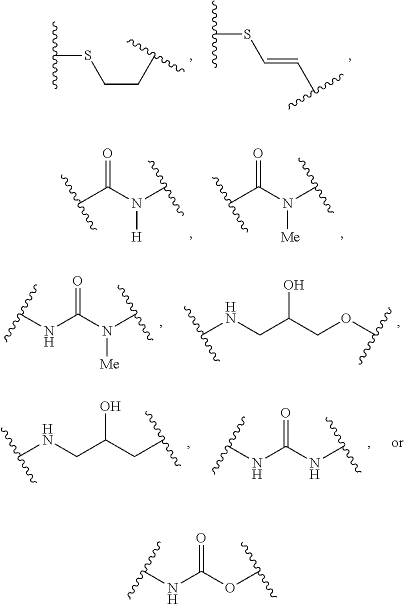

where each L2 and L3, where present, is independently a methylene spacer between 1 and 8 carbons in length e.g. —[(CH₂)₁₋₈]—;
and IG1 is a polyionic group selected from the following:

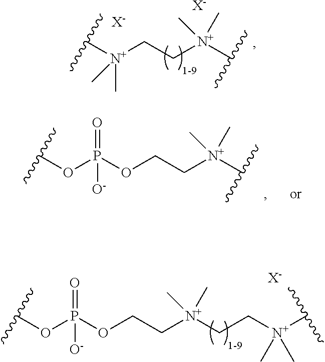

each X− is an anion selected from Cl⁻, Br⁻, I⁻, F⁻, SO₄²⁻, CO₃²⁻, PO₄³⁻, CH₃SO₃⁻, CF₃SO₃⁻, BF₄⁻, TsO⁻, AcO⁻, BzO⁻ and NTf₂⁻. In another embodiment the present invention describes a class of polyionic silanization reagents of the formula III:

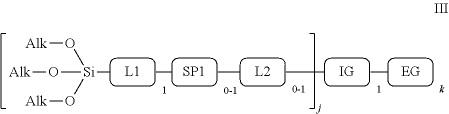

wherein:
j is 1 or 2; k is 0 or 1, such that the values for j and k satisfy the condition that j+k=2;
Alk is selected from Me, Et, n-Pr, i-Pr, n-Bu, sec-Bu or t-Bu;
L1 is a methylene chain between 2 and 10 carbons in length e.g. —[(CH₂)₂₋₁₀]—
SP1, where present, is a spacer selected from:

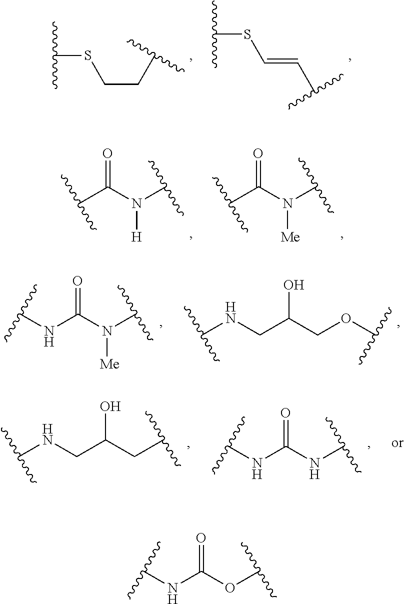

L2, where present, is a methylene chain between 1 and 8 carbons in length, e.g. —[(CH₂)₁₋₈]—;
IG is a polyionic group selected from the following:

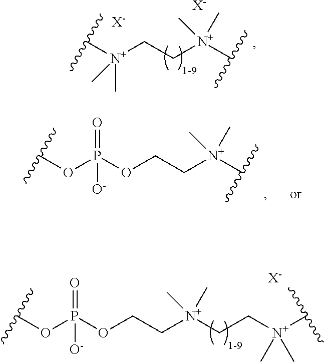

EG is the end group selected from methyl, —[(CH₂CH₂O—)₁₋₃₀]-Me, —[(CH₂CH₂O)₁₋₃₀]—H, or a linear n-alkyl chain between 2 and 8 carbons in length; and
each X− is an anion selected from Cl⁻, Br⁻, I⁻, F⁻, SO₄²⁻, PO₄³⁻, CO₃²⁻, CH₃SO₃⁻, CF₃SO₃⁻, BF₄⁻, TsO⁻, AcO⁻, BzO⁻ and NTf₂⁻.

In another embodiment, the present application describes polyionic surface binding reagents that do not require a trialkoxysilyl group to bind to the surface, of the formula IV:

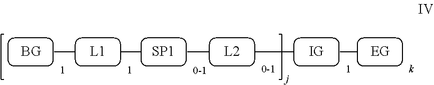

wherein:
BG is selected from:

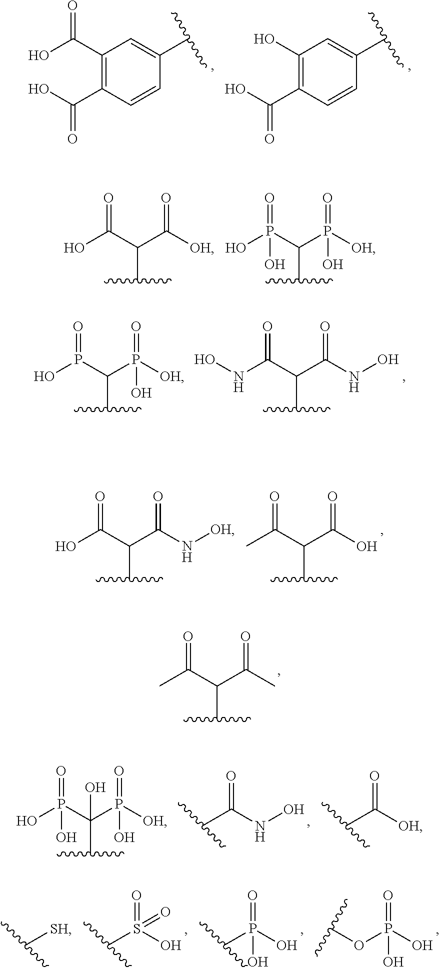

j is either 1 or 2; k is either 0 or 1, such that the values for j and k satisfy the condition that j+k=2;
L1 is a methylene chain between 2 and 10 carbons in length e.g. —[(CH₂)₂₋₁₀]—;
SP1, where present, is a spacer selected from

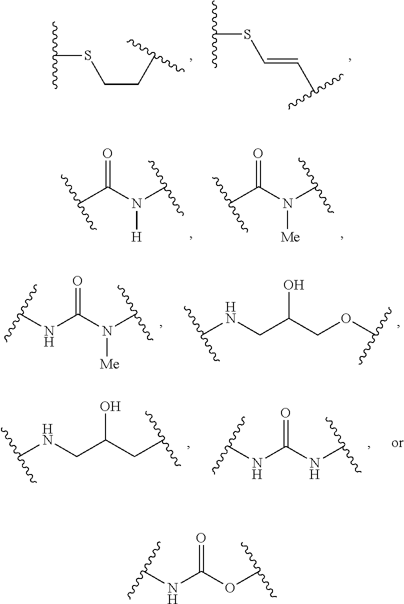

L2, where present, is a methylene chain between 1 and 8 carbons in length e.g. —[(CH₂)₁₋₈]—;
IG is a polyionic group selected from the following:

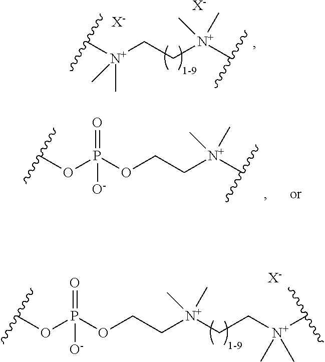

EG is the end group selected from methyl, —[(CH₂CH₂O—)₁₋₃₀]-Me, —[(CH₂CH₂O)₁₋₃₀]—H, or a linear n-alkyl chain between 2 and 8 carbons in length;
each X− is an anion selected from Cl⁻, Br⁻, I⁻, F⁻, SO₄²⁻, PO₄³⁻, CO₃²⁻, CH₃SO₃⁻, CF₃SO₃⁻, BF₄⁻, TsO⁻, AcO⁻, BzO⁻ and NTf₂⁻.

In one variation of the formula IV, BG is selected from the group consisting of:

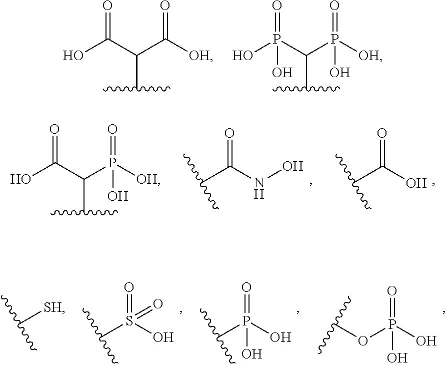

In one embodiment, the molecules of formulae III and IV may be used as an antifouling coating on surfaces such as SiO₂, glass, calcium oxide, enamel, bone, tooth enamel, tooth dentin, hydroxyapatite, kaolin or zirconia. In some aspects, the BG's of formula IV react with the surface minerals and/or metals to form strong ionic and/or hydrogen bonds. Once applied to the surface, the enhanced hydrophilicity of the multiple ionic residues helps to attract a strong hydration layer (i.e., water) rendering the surface resistant to the biofouling process.

In other embodiments, the molecules of formulae III and IV may be used to form an antifouling surface on metallic surfaces such as, aluminum, copper, chrome, chrome-cobalt, titanium, zinc, iron, bronze, steel, stainless steel, high carbon steel, tin, indium-tin. In other aspects, the coating on such metals may form a passivating layer and prevent corrosion of the substrate.

In one variation, when j=2 for the molecules of formulae III and IV, the molecules form polyionic loops, i.e., two points of attachment, to the surface owing to the presence of two groups which each bind to the surface, being connected to each other through the chain by the polyionic moiety. In one aspect, these molecules bear structural similarity to gemini surfactants. In another aspect, the presence of multiple ionic residues helps to drive self aggregation upon the surface.

The molecules of formulae III, and IV, may be used in an appropriate solvent to form priming solutions that act as antifouling primers, anticorrosion primers, and or hydrophilicity-enhancing primers. The molecules of formula III may be used to form the surface coatings of formulas I and II according to procedures given herein and referenced elsewhere in this disclosure.

The surface coating, priming or deposition of the compounds of the present application may be performed using standard methods known in the art, with the exception of the particular improved procedures and formulations developed and disclosed herein. For dental and medical applications, the primer may be provided in a solvent, such as water, methanol, ethanol, isopropanol, acetone, or mixtures thereof. For dental applications, the same solvent, solvent blends, or different solvent may be used to wash the surface of the tooth or enamel. In certain applications, when the solvent is water, the process provides an environmentally friendly and effective process. In one application, the solution employed may be used at a neutral pH, or may be maintained in acidic conditions, at a pH<7, pH<6 or pH<5. The pH may be adjusted using an acid, such as phosphoric acid, hydrochloric acid, acetic acid or sulfonic acid.

Depending on the type of application or the type of compound or primer employed, the pH of the solution may be >pH 5, >pH 6, >pH 6.5 or >pH 7. The solution may be degassed using an inert gas or using vacuum or a combination thereof.

Depending on the particular application, the concentration of the primer in the solution may be prepared at different concentrations and concentration ranges, such as a 0.0001 wt. % to 20 wt. %, 0.0001 wt. % to 15 wt. %, 0.0001 wt. % to 10 wt. %, about 0.001 wt. % to 10 wt. %, about 0.01 wt. % to 10 wt. %, about 0.1 wt. % to 10 wt. % or at about 0.1 wt. % to 5 wt. %; at 0.0001 wt %, 0.001 wt. %, 0.01 wt %, 0.1 wt %, 1.0 wt %, 5 wt %, 10 wt %, 15 wt %, 20 wt %, 25 wt % or more, in a solvent or solvent mixture.

In one embodiment, the solution may be applied onto a surface, such as a mineral, metal, and/or metal oxide surface for a period of time to allow the compound or primer (e.g., formulae I to IV) to set up or otherwise adsorbed to or adhere to the surface. Depending on the nature of the surface and the structure of the compound, adhesion of the compound to the surface may take less than about 30 minutes, less than 10 minutes, less than 5 minutes, less than 3 minutes, less than 2 minutes or less than about 1 minute. Once the primer is adsorbed to the surface, any excess primer may be removed from the surface by washing or rising with a solvent or solvent mixture. For certain applications, the solvent or solvent mixture may be water, ethanol, or a mixture of water and ethanol solution. Depending on the desired application, the surface with the adsorbed primer may be dried using air, heat or a combination thereof until the desired dryness is achieved. The solvent or solvent mixture employed in the primer solution and/or as a washing solvent may include water, methanol, ethanol, propanol, isopropanol, acetone, methylethyl ketone, hexane, petroleum ether, diethyl ether, MTBE, cyclohexane, heptane, toluene, xylenes, THF, DMF, MeCN, Me-THF, CH₂Cl₂, CHCl₃, and N-methylpyrrolidone, or various mixtures thereof. In one variation, the solvent or solvent mixtures is methanol, ethanol, acetone and CH₂Cl₂, or mixtures thereof. In certain applications, the solvent is water, or a mixture of the solvent(s) with water, and the process provides an environmentally friendly and effective process.

The thickness of the adhered/adsorbed layer may be about 0.5-50 nm, 0.1-40 nm, 0.1-30 nm, 0.1-20 nm, 0.1-10 nm, 0.1-5 nm or 0.1-3 nm. For deposition of the solution comprising the compound or primer of the present application, the thickness will depend on the nature of the compound and the desired thickness of the layer and the nature of the application. For the preparation of SAMs, the thickness of the adhered or adsorbed layer may be less than for other self-assembled layers with the desired thickness. Optionally, the surface comprising a first layer may be completely dried before applying second layer or subsequent layers.

The molecules and coatings of formulae I-II and primers III and IV, may be used in combination with other biocidal agents and surfactants, to improve their antifouling properties.

In some embodiments, the polyionic silanes of formula I may be polymerized by addition of water to the compounds of formula I to generate trihydroxysilanes, polysiloxanes and polysequisiloxanes. The resulting polymers may be applied to various surfaces to render them biocidal/antiviral/antifouling. In one embodiment the compounds of formula I, are dissolved in ethanol and then diluted with distilled water to hydrolyze alkoxy-residues and make a 5 wt % solution of polymers. The resulting polymers may then be diluted with water and ethanol to make solutions containing 0.01-0.1 wt %, 0.1-0.5 wt %, 0.5-1.0 wt %, or 1.0-4.99 wt % polymer. Solutions of such polymers may then be applied to various surfaces such as plastics, metals, fabrics, whereupon the solution is evaporated and optionally heat cured to obtain biocidal/antiviral/antifouling surfaces.

### Example 1: N¹,N¹,N³,N³-tetramethyl-N¹,N³-bis(3-(trimethoxysilyl)propyl)propane-1,3-diaminium Iodide

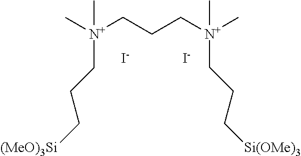

20 mmol of (N,N-dimethylaminopropyl)trimethoxysilane) was dissolved in 100 ml of anhydrous MeCN under inert atmosphere in a 250 ml heavy walled schlenk flask, fitted with a football shaped stir bar, and rubber septa. 10 mmol of 1,3 diiodopropane was added slowly via syringe, whereupon the schlenk valve was sealed and the flask was heated to 70° C. in an oil bath for 72 h. The stir bar was removed and the volatiles were removed first by rotary evaporation and then by high vacuum, to obtain N¹,N¹,N³,N³-tetramethyl-N¹,N³-bis(3-(trimethoxysilyl)propyl)propane-1,3-diaminium iodide as a yellow waxy froth of bubbles.

### Example 2: N₁,N¹,N³,N³-tetramethyl-N¹,N³-bis(8-(trimethoxysilyl)octyl)propane-1,3-diaminium Bromide

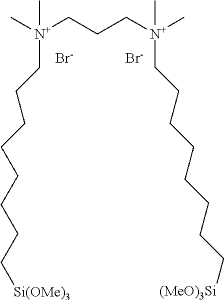

The title compound is obtained as described for the N¹,N¹,N³,N³-tetramethyl-N¹,N³-bis(3-(trimethoxysilyl)propyl)propane-1,3-diaminium iodide, reacting N,N-dimethylaminooctyl)trimethoxysilane with 1,3, dibromopropane.

### Example 3: Representative Procedures for Hydroxylating a PDMS Surface

It is well known in the literature that PDMS surfaces, once oxidized, are susceptible to hydrophobic recovery. Therefore, once oxidized, they must be immediately reacted with the appropriate silanization reagent.

Method A (pirhana solution): A small section of PDMS is submerged in an aqueous containing 20% v/v concentrated sulfuric acid with stirring. Very cautiously, an equivalent volume of 30% H₂O₂ (equivalent in volume to amount of H₂SO₄) is added very slowly dropwise over 30 minutes to the solution with the submerged PDMS. The mixture is stirred for an additional 30 minutes whereupon the PDMS is carefully removed and the interior and exterior surfaces are rinsed repeatedly with distilled water then by anhydrous methanol, ethanol or acetone.

Method B (plasma cleaning): A small section (about 2×2 cm) of a PDMS sheet tube was treated with oxygen plasma (Harrick air-plasma cleaner, PDC-32G) at a power of 18 Watts and a vacuum level of 0.3 Torr for 30 seconds.

### Example 4: Generating a Molecularly Well Defined, Polyionic Antibiofouling Coating Upon a PDMS Surface

A 5% wt/v solution of N¹,N¹,N³,N³-tetramethyl-N¹,N³-bis(3-(trimethoxysilyl)propyl)propane-1,3-diaminium iodide obtained as described in example 2, is prepared in anhydrous methanol, and the freshly hydroxylated PDMS surface from example 3 is submerged in said solution under inert atmosphere and left to react with gentle stirring for 2-24 hours. Once complete the surface is removed from the solution and rinsed repeatedly with methanol. To exchange the iodide (or other non-chlorine) anions for chloride ions the surface, or when the surface comprises a medical device such as a catheter, is subsequently rinsed repeatedly with a 0.1N solution of NaCl in distilled water, then with pure DI water, and finally methanol before being dried under nitrogen.

Alternative method (after plasma cleaning): The oxidized PDMS samples were immediately removed from the plasma cleaner and dipped in a solution of 5 mM of N¹,N¹,N³,N³-tetramethyl-N¹,N³-bis(3-(trimethoxysilyl)propyl)propane-1,3-diaminium iodide in anhydrous methanol with (optionally) 0.2% of added deionized water. After 10 h incubation at a room temperature, the PDMS samples were rinsed with methanol, then repeatedly with a 0.1 N solution of NaCl to exchange iodide for chloride ions, then with pure DI water, and finally methanol before being cured at 80° C. for 30 min.

### Example 5. Preparation of a Zwitterionic, Thiol-Reactive Substrate, for Generation of Antibiofouling Coatings

5 ml of 5-hexene-1-ol (41.6 mmol, 1 equiv) was dissolved in 100 ml of anhydrous Et₂O, in a 250 ml round bottom flask under inert atmosphere fitted with a stir bar and rubber septa. 6.1 ml of Et₃N (43.7 mmol, 1.05 equiv) was added via syringe and the flask was cooled to −10° C. in an ice/salt bath. 4.02 ml of 2-chloro-1,3,2-dioxaphospholane 2-oxide (43.7 mmol, 1.05 equiv) was then added slowly dropwise at −10° C. with concomitant formation of Et₃N.HCl precipitate and the solution was left to react at −10° C. for 30 minutes before being gradually warmed to ambient temperature over 2 hours. The contents of the flask were then diluted with ether and the amine hydrochloride salt was filtered off over diatomaceous earth in a fritted funnel into a round bottom flask and the volatiles were removed by rotary evaporation to obtain the crude 2-(hex-5-en-1-yloxy)-1,3,2-dioxaphospholane 2-oxide as a pale yellow oil which was used immediately in the next step.

The crude from the preceding step was then dissolved in 25 ml of anhydrous methanol and transferred to a flame dried, argon purged, heavy walled, schlenk flask fitted with a stir bar. 26 ml of 25% Me₃N in MeOH (ca 3.2 M), was then added to the schlenk flask which was then sealed and heated at 45-55° C. until TLC indicated complete disappearance of the intermediate oxaphospholane. The stir bar was removed and the volatiles were then removed by rotary evaporation to crude zwitterionic coupling agent which was then purified by chromatography on ethylated silica (Analtech Unibond C2 150 Å pore size, 35-75 micron, Catalog #B08010) to obtain 2.69 g of essentially pure hex-5-en-1-yl (2-(trimethylammonio)ethyl) phosphate as a white waxy compound.

### Example 6. Coupling of Thiol Functionalized PDMS Surfaces with Alkene Functionalized Zwitterionic Coupling Agents to Generate an Antibiofouling Coating

A PDMS surface was hydroxylated as described in Example 3, then functionalized with 3-(metcaptopropyl)trimethoxysilane in methanol analogously to the silanization procedure given in Example 4. The thiol functionalized PDMS surface was then submerged in a 5% solution of freshly prepared hex-5-en-1-yl (2-(trimethylammonio)ethyl) phosphate in distilled water, methanol, and/or ethanol, also containing 2 mol % (relative to phosphate) of DMPA (2,2-dimethoxy-2-phenylacetophenone) as a photoinitiator and irradiated with UV light for 24 hours to achieve a thiol-ene reaction between the surface and zwitterionic coupling agent. Excess coupling agent was washed away with repeated rinsing with distilled water, and the surface was then dried to obtained the desired zwitterionic antibiofouling PDMS surface.

### Example 7. Preparation of an Unsymmetrical Dicationic, Thiol-Reactive Substrate, for Generation of Antibiofouling Coatings

To a flame dried round bottom flask fitted with a stir bar, rubber septa, and argon needle was added 50 ml of anhydrous CH₂Cl₂ followed by 37.5 ml of anhydrous TMEDA (tetramethylethylenediamine). The solution was then placed in a −78° C. bath (dry ice/acetone) and stirred gently for 15 minutes while allowing the temperature to equilibrate. The rubber stopper was then removed and replaced with a dry, pressure equalizing addition funnel containing 15.5 ml of methyl iodide dissolved in 25 ml of anhydrous CH₂Cl₂. The solution containing methyl iodide was then allowed to drip into the flask at a rate of about 1 drop/second. Once the addition was complete the mixture was left to react overnight with concomitant warming of the cooling bath to room temperature. The mixture was then diluted with hexanes to assist in precipitation of the product with stirring and the powder was collected on a Buchner funnel, washing the powder successively with 3×100 ml hexanes, then 3×50 ml acetone and dried under vacuum. The powder was then transferred to a round bottom flask whereupon residual volatiles were removed under high vacuum overnight to obtain 61.3 grams (95% of theoretical) of 2-(dimethylamino)-N,N,N-trimethylethanaminium iodide as a white to tan powder.

To a flame dried heavy walled schlenk flask fitted with a football shaped stir bar under argon was added ca. 30 ml of anhydrous MeCN, followed by 5.94 g (23 mmol, 1 equiv) of the preceding mono-quaternary bis-amine, and 3 ml (25.3 mmol, 1.1 equiv) of 5-bromo-1-pentene. The schlenk valve was sealed and then the flask was heated to 70° C. in an oil bath whereupon the solids became fully dissolved. The mixture was left to react with stirring at 70° C. for 72 hours whereupon there was observed formation of a large amount of white precipitate. The heating bath was removed and the mixture was then filtered while still warm to obtain 5.90 g of N¹,N¹,N₁,N²,N²-pentamethyl-N²-(pent-4-en-1-yl)ethane-1,2-diaminium bromide iodide as an off white powder.

### Example 8. Preparation of N¹,N¹,N²,N²-tetramethyl-N¹,N²-di(pent-4-en-1-yl)ethane-1,2-diaminium Bromide as a Symmetrical, Dicationic, Thiol-Reactive Substrate, for Generation of Antibiofouling Coatings

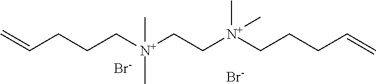 [SMILES: C=CCCC[N+](C)(C)CC[N+](C)(C)CCCC=C ; [Br-] ; [Br-]]

To a flame dried heavy walled schlenk flask fitted with a football shaped stir bar under argon was added ca. 30 ml of anhydrous MeCN, followed by 5 ml (42.2 mmol, 2.2 equiv) of 5-bromo-1-pentene, and 2.88 ml (19.2 mmol, 1 equiv) of TMEDA. The schlenk valve was sealed and then the flask was heated to 70° C. in an oil bath whereupon the mixture was left to react with stirring at 70° C. for 72 hours. Once the indicated time had elapsed the mixture was transferred to a round bottom flask and the volatiles were removed under reduced pressure to obtain a gummy solid which was first triturated with ether, then extracted with hot acetone to obtain a solid, which was then washed with additional hexanes on a Buchner funnel to obtain 3.45 g (43.4% of theoretical) of pure N¹,N¹,N²,N²-tetramethyl-N¹,N²-di(pent-4-en-1-yl)ethane-1,2-diaminium bromide. The 1H-NMR spectrum of the product in CDCl₃ is shown below:

### Example 9: Thiol-Ene Reaction Between Dicationic Alkene to Form a Dicationic Silanization Reagent

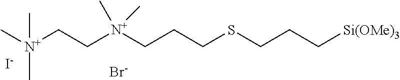

1 equivalent of N¹,N¹,N¹,N²,N²-pentamethyl-N²-(pent-4-en-1-yl)ethane-1,2-diaminium bromide iodide, is dissolved in a minimum amount of anhydrous MeOH, along with 1 equivalent of 3-(metcaptopropyl)trimethoxysilane and 2 mol % of Irgagure 651 (2,2-dimethoxy-1,2-diphenylethanone). The reaction mixture is then capped with a septum and purged with argon. The flask is then placed next to a 15 W, 18″-long blacklight having a total UV output of 2.6 W and Xmax=368 nm. The flask was positioned so that one side rested against the center of the bulb. Both the flask and blacklight is wrapped in aluminum foil and the reaction mixture is irradiated for ca. 24 hours whereupon concentration under reduced pressure affords the desired N¹,N¹,N¹,N²,N²-pentamethyl-N²-(5-((3-(trimethoxysilyl)propyl)thio)pentyl)ethane-1,2-diaminium bromide iodide which is protected from light and moisture.

### Example 10: Preparation of a Hybrid Zwitterionic/PEG Containing Silanization Reagent

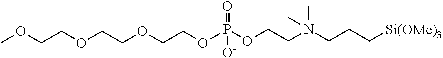

5 ml of Triethylene Glycol Monomethyl Ether (31.97 mmol, 1 equiv) is dissolved in ca 100 ml of anhydrous Et₂O/THF (1:1 v:v), in a 250 ml round bottom flask under inert atmosphere fitted with a stir bar and rubber septa. 4.68 ml of Et₃N (33.56 mmol, 1.05 equiv) is added via syringe and the flask is cooled to −10° C. in an ice/salt bath. 3.09 ml of 2-chloro-1,3,2-dioxaphospholane 2-oxide (33.6 mmol, 1.05 equiv) is then added slowly dropwise at −10° C. with concomitant formation of Et₃N.HCl precipitate and the solution was left to react at −10° C. for 30 minutes before being gradually warmed to ambient temperature over 2 hours. The contents of the flask were then diluted with Ether and the amine hydrochloride salt was filtered off over diatomaceous earth in a fritted funnel into a round bottom flask and the volatiles were removed by rotary evaporation to obtain the crude dioxaphospholane which is used immediately in the next step.

The crude from the preceding step is then dissolved in ca. 25 ml of anhydrous methanol and transferred to a flame dried, argon purged, heavy walled, schlenk flask fitted with a stir bar. 1.05 equivalents of (N,N-dimethylaminopropyl)trimethoxysilane is then added, the flask is sealed and left to react at 45° C. until TLC shows complete disappearance of the intermediate oxaphospholane. The stir bar is removed and the volatiles are removed by rotary evaporation to crude zwitterionic coupling agent which is used without further purification.

### Example 11: Synthesis of 3-(2,8,9-trioxa-5-aza-1-silabicyclo[3.3.3]undecan-1-yl)-N,N-dimethylpropan-1-amine (3-dimethylaminopropyl-silatrane)

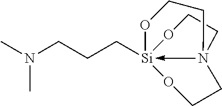 [SMILES: CN(C)CCC[Si-]123OCC[N+]1(CCO2)CCO3]

The title compound was synthesized from N,N′dimethylaminopropyltrimethoxy silane as follows: To a flame dried 2-neck round bottom flask fitted with a PTFE coated stir bar, a reflux condenser, and a Dean Stark trap. 250 ml was added ca. 150 ml of anhydrous toluene. 6.28 ml (7.06 g, 47.3 mmol) of anhydrous triethanolamine was added to the flask via syringe followed by 9.95 ml (9.46 g, 45.6 mmol) of N,N′dimethylaminopropyl trimethoxysilane and the solution was stirred. The flask was placed into an oil bath and the mixture was heated to 80° C. and stirred at 80° C. under inert atmosphere overnight. The mixture was then heated to reflux and once the dean stark became initially filled the dean stark was drained and mixture was refluxed for 8 h with periodic draining of the dean stark trap every hour or so to remove methanol. The mixture was distilled down to a volume of approx. 30 ml and the flask was removed from the oil bath and let cool to ambient temperature. The stir bar was removed and the volatiles were removed by rotary evaporation, the product was precipitated from the residue by addition of hexanes, and the hexanes was decanted off, whereupon the residue was recrystallized from acetone to obtain the title compound as a white powder. Note: the title compound may also be obtained by stirring the mixture at room temperature overnight with a catalytic amount (about 1-5 mol % of sodium methoxide or sodium hydroxide) followed by refluxing to distill off methanol and toluene.

### Example 12: Synthesis of N-(3-iodopropyl)-N,N-dimethyloctadecan-1-aminium Iodide

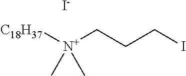

3 ml of 1,3 diiodopropane was added to a flame dried 100 ml round bottom flask fitted with a rubber septa and PTFE coated stir bar followed by 60 ml of anhydrous acetone. The mixture was stirred and 2.98 g of N,N′-dimethylamino-octadecane was added via syringe whereupon the flask was wrapped in tin foil to protect from light and stirred in the dark for 48 h. After 48 h stirring was ceased and the mixture was cooled to 0° C. and the product was collected by vacuum filtration on a Buchner funnel rinsing the filter cake with ice cold acetone to obtain the title compound as a white powder which was stored in tightly sealed amber vials protected from light and moisture.

### Example 11: Synthesis of N¹-(3-(2,8,9-trioxa-5-aza-1-silabicyclo[3.3.3]undecan-1-yl)propyl)-N¹,N¹,N³,N³-tetramethyl-N-octadecylpropane-1,3-diaminiumiodide

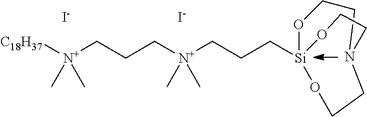

1.5 grams of the preceding iodide along with 823 mg of N,N′dimethylaminosilatrane was added to a flame dried Schlenk-bomb type flask fitted with a PTFE stir bar under Argon atmosphere. 10 ml of Anhydrous MeCN was added to the flask followed by 30 ml of anhydrous DMF and the schlenk-valve was sealed and the mixture was heated to 85° C. with stirring for 72 h. The mixture was transferred to a round bottom flask and the volatiles were removed by rotary evaporation to obtain a viscous oil, which solidified upon addition of acetone. The mixture was triturated with acetone and the organics were decanted off to obtain a white powder which was further rinsed with additional acetone and dried on high vacuum to obtain 1.2 g of the title compound.

Several of the aforementioned compounds were used to coat PDMS surfaces, by either direct silanization, or by silanization with mercaptopropyltrimethoxysilane (MPTMS) and subsequent thiol-ene reaction to obtain a library Gemini-surfactant inspired polyionic surface coatings as follows.

Representative library of polyionic alkenes and silanization reagents

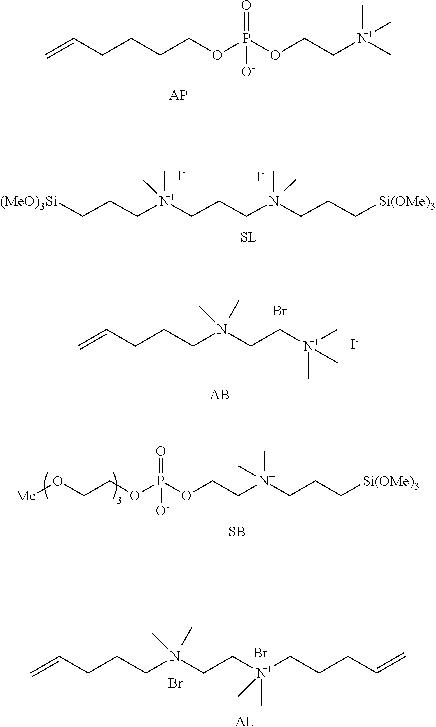

Water droplet contact angle measurements of a representative library of gemini-surfactant inspired polyionic surface coatings are shown below.

As seen above native silicone PDMS (poly-dimethylsiloxane) surfaces are hydrophobic with static water-droplet contact angle measurements around 80°, while 02-plasma treatment and subsequent silanization with thiol-modified silane MPTMS both modestly increase hydrophilicity. Treating surfaces with both the silanizing loop SL, and brush, SB, reagents increases the hydrophilicity somewhat compared to the native PDMS. Silanization with MPTMS and subsequent thiol-ene reaction with alkenes AL, AB and AP gives rise to highly hydrophilic surfaces with contact angles of between 40-25°.

Data from a crystal-violet dynamic biofilm assay indicates the coatings of the present invention are useful to prevent biofouling. After incubating surfaces for 3 days at 37° C. with S. aureus in tryptic-soy broth (TSB) media and rinsing away unadhered bacteria, unmodified silicone surfaces are fully colonized (left) while a gemini-AB-coated surface (right) shows almost no living bacteria attached to the surface as shown below:

The image above shows images of S. aureus attachment to unmodified silicone (left); vs gemini-AB-modified silicone (right) after 3 days incubation, visualized with crystal-violet.

### Example 12: Synthesis of a Polyionic Gemini-Inspired Antimicrobial Silicone Polymer

8.64 ml (76.8 mmol, 2.5 equiv) of TMEDA is dissolved in 100 ml of anhydrous acetone whereupon 10.5 ml (30.7 mmol, 1 equiv) of stearyl-chloride (1-chloro-octadecane) is added via syringe. The solution is heated in a sealed flask at 80° C. for 96 h then cooled to ambient temperature and the volatiles are removed by rotary evaporation under reduced pressure. Excess TMEDA and colored impurities are removed by rinsing the residue repeatedly with diethyl ether to obtain N-(2-(dimethylamino)ethyl)-N,N-dimethyloctadecan-1-aminium chloride.

4 grams of the preceding chloride (10 mmol) and 1.82 ml (10 mmol) of (3-Chloropropyl)trimethoxysilane are dissolved in anhydrous Methanol up to a volume of 10 ml and heated in a dry, sealed flask under inert atmosphere at 80° C. for 7 days or until aliquots of the reaction mixture showed no further change in the consumption of silane resulting in a solution containing approx 60% wt of of N¹,N¹,N²,N²-tetramethyl-N¹-octadecyl-N²-(3-(trimethoxysilyl)propyl)ethane-1,2-diaminium chloride in MeOH which may be used for subsequent covalent surface modification or converted into antimicrobial silicone-polymers.

### Example 13: Polymerization of an Antimicrobial Polyionic Silane by Addition of Water

10 ml of the preceding solution is then added to 110 ml of distilled water in a round bottom flask and stirred gently for 1 week at 40° C. to hydrolyze/polymerize alkoxylsilyl residues and generate a 5% wt/v aqueous solution of N¹,N¹,N²,N²-tetramethyl-N¹-octadecyl-N²-(3-(trihydroxysilyl)propyl)ethane-1,2-diaminium chloride hereafter termed 18-2-3-G-Sil-Cl, as a mixture of trihydoxysilanes, polysiloxanes and sesquisiloxanes.

### Example 13: Antimicrobial and Fast Evaporating Ethanolic Solution of 18-2-3-G-Sil-Cl

300 ml of the 5% wt 18-2-3-G-Sil-Cl is added to 700 ml of 200 proof EtOH to make a 0.5% wt solution in 70% EtOH/Water.

### Example 14: Antimicrobial Aqueous Solution of 18-2-3-G-Sil-Cl

300 ml of the 5% wt 18-2-3-G-Sil-Cl is added to 700 ml of distilled to make 1 L of a 0.5% wt solution in water.

### Example 15: Conversion of a Cotton Facemask into an Antimicrobial-Polymer Coated Cotton Facemask

A cotton facemask is submerged into the solution of Example 13, and let evaporate to dryness at ambient temperature. The surface of the resulting facemask is biocidal/antiviral towards microbes and enveloped viruses that settle upon its surface.

### Example 16: Conversion of Cotton Fabric into a Covalently-Modified Antimicrobial Cotton Fabric

A piece of cotton fabric is submerged into a 0.5 mM Methanolic solution of Example 9, and let evaporate to dryness at ambient temperature. The surface of the resulting cotton fabric is biocidal/antiviral towards microbes and enveloped viruses that settle upon its surface.

### REFERENCES

1. Wallace, A.; Albadawi, H.; Patel, N.; Khademhosseini, A.; Zhang, Y. S.; Naidu, S.; Knuttinen, G.; Oklu, R. Cardiovascular Diagnosis and Therapy, 2017, 7, S246-S257.
2. Mermel, L. A. “Prevention of intravascular catheter-related infections.” Ann Intern Med 2000, 132, 391-402.
3. Rodney M.; Donlan, J.; Costerton, W. “Biofilms: Survival mechanisms of Clinically Relevant Microorganisms.” Clinical Microbiology Reviews, 2002, 15, 167-193.
4. Ding, X.; Yang, C.; Lim, T. P.; Hsu, L. Y.; Engler, A. C.; Hedrick, J. L.; Yang, Y-Y. “Antibacterial and antifouling catheter coatings using surface grafted PEG-b-cationic polycarbonate diblock copolymers.” Biomaterials, 2012, 33, 6593-6603.
5. Smith, R. S.; Zhang, Z.; Bouchard, M.; Li, J.; Lapp, H. S.; Brotske, G. R.; Lucchino, D. L.; Weaver, D.; Roth, L. A.; Coury, A.; et al. “Vascular catheters with a nonleaching poly-sulfobetaine surface modification reduce thrombus formation and microbial attachment.” Sci. Transl. Med. 2012, 4, 153-132.
6. Vaterrodt, A.; Thallinger, B.; Daumann, K.; Koch, D.; Guebitz, G. M.; Ulbricht, M. “Antifouling and Antibacterial Multifunctional Polyzwitterion/Enzyme Coating on Silicone Catheter Material Prepared by Electrostatic Layer-by-Layer Assembly.” Langmuir, 2016, 32, 1347-1359.
7. Papra, A.; Bernard, A.; Juncker, D.; Larsen, N. B.; Michel, B.; Delamarche, E. Langmuir, 2001, 17, 4090-4095.
8. Qiu, W.; Wu C.; Wu, Z. “Surface Modification of PDMS in Microfluidic Devices.” in: Concise Encyclopedia of High Performance Silicones. 2014, JohnWiley & Sons, Inc. Hoboken, N.J., and Scrivener Publishing LLC, Salem, Mass.
9. Menger, F. M.; Keiper. J. S. “Gemini Surfactants” Angew. Chem. Int. Ed. 2000, 39, 1906-1920.
10. Huang, C-J.; Chang, Y-C. “In Situ Surface Tailoring with Zwitterionic Carboxybetaine Moieties on Self-Assembled Thin Film for Antifouling Biointerfaces” Materials, 2014, 7, 130-142.
11. Hu, G.; Emrick, T. “Functional Choline Phosphate Polymers” J. Am. Chem. Soc. 2015
12. For peptide coupling procedures with amino-silylated substrates, refer to the Thermo-Scientific technical bulletin: “Tech Tip #1: Attach a protein onto glass, silica, or quartz surface using a cleavable crosslinker” Thermo-Scientific, 2008, available at http://www.thermo.com/pierce, Accessed: 15 Oct. 2018.
13. Levering, V.; Cao, C.; Shivapooja, P.; Levinson, H.; Zhao, X.; Lopez, G. P. “Urinary catheter capable of repeated on-demand removal of infectious biofilms via active deformation” Biomaterials, 2016, 77, 77-86.
14. Ahn, B. K.; Das, S.; Linstadt, R. T. H.; Kaufman, Y.; Martinez-Rodriguez, N. R.; Mirshafian, R.; Kesselman, E.; Talmon, Y.; Lipshutz, B. H.; Israelachvili, J. N.; Waite, J. H. “High-performance mussel-inspired adhesives of reduced complexity” Nat. Commun. 2015, 6, 8663.
15. Das, S.; Lee, B. H.; Linstadt, R. T. H; Cunha, K.; Kaufman, Y.; Levine, Z. A.; Lipshutz, B. H.; Lins, R. D.; Shea, J-E.; Heeger, A. J.; Ahn B. K. “Molecularly-smooth Self-Assembled Monolayer for High-Mobility Organic Field-Effect Transistors” Nano Lett., 2016, 16, 6709.

1. A polyionic surface coating of the formula I: 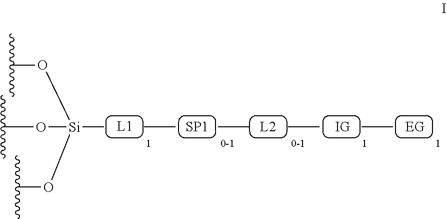
wherein:
the silyloxy portion depicted above (—(O)₃Si—) is covalently bonded to a surface;
L1 is —[(CH₂)₂₋₁₀]—;
SP1, where present, is a spacer selected from 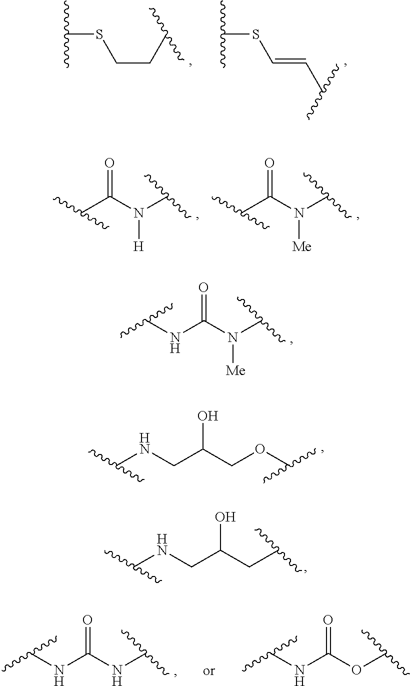
L2, where present is —[(CH₂)₁₋₈—]— or —[(CH₂CH₂O)₁₋₃₀]—;
IG is a polyionic group selected from the following: 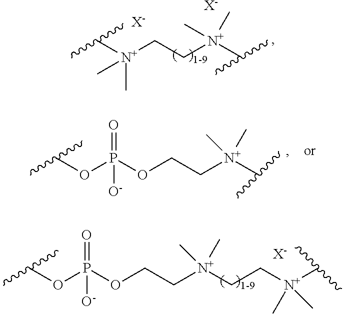
EG is the end group selected from methyl, —[(CH₂CH₂O)₁₋₃₀]-Me, —[(CH₂CH₂O)₁₋₃₀]—H, or a linear n-alkyl chain between 2 and 20 carbons in length.
each X⁻ is independently an anion selected from Cl⁻, Br⁻, F⁻, SO₄²⁻, PO₄³⁻, CO₃²⁻, CH₃SO₃⁻, CF₃SO₃⁻, BF₄⁻, TsO⁻, AcO⁻, BzO⁺ and NTf₂⁻.

2. A polyionic surface coating of the formula II: 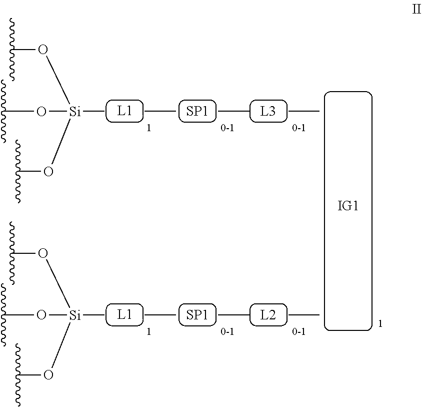
wherein:
the group —(O)₃Si— is covalently bonded to a surface;
each L1, L2 and L3 is independently —(CH₂)₂₋₁₀— or —[(CH₂CH₂O)₁₋₃₀]—;
each SP1 and SP2 is a spacer independently selected from: 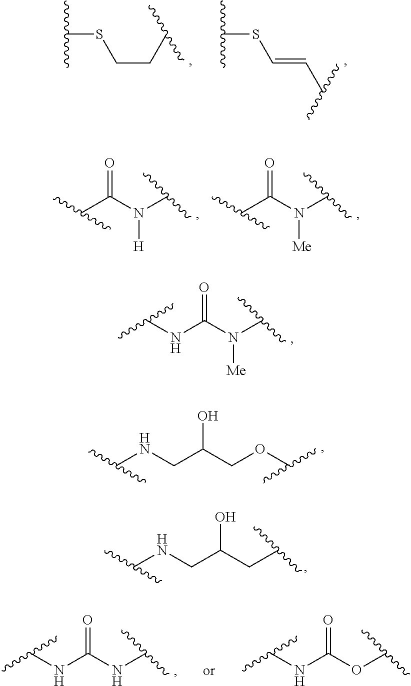
IG1 is a polyionic group selected from: 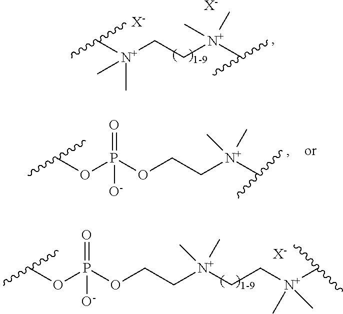
each X⁻ is independently an anion selected from Cl⁻, Br⁻, I⁻, F⁻, SO₄²⁻, CO₃²⁻, PO₄³⁻, CH₃SO₃⁻, CF₃SO₃⁻, BF₄⁻, TsO⁻, AcO⁻, BzO⁻ and NTf₂⁻.

3. A polyionic silanization reagent of the formula III: 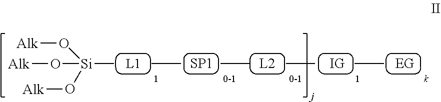
wherein:
j is either 1 or 2; k is either 0 or 1, such that the values for j and k satisfy the condition that j+k=2;
Alk is methyl, ethyl, n-Pr, i-Pr, n-Bu, sec-Bu or t-Bu (or H after hydrolysis);
L1 is —[(CH₂)₂₋₁₀]— or —[(CH₂CH₂O)₁₋₃₀]—;
SP1 is a spacer selected from: 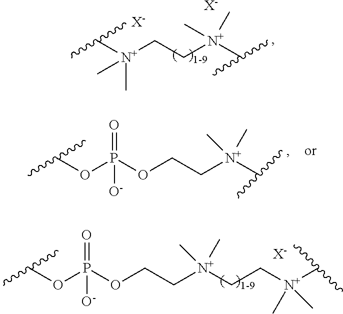
L2 is —[(CH₂)₁₋₈]— or —[(CH₂CH₂O)₁₋₃₀]—;
IG is a polyionic group selected from the following: 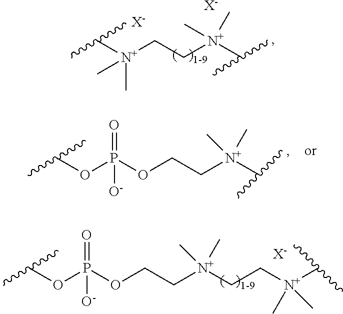
EG is the end group selected from methyl, —[(CH₂CH₂O)₁₋₃₀]-Me, —[(CH₂CH₂O)₁₋₃₀]—H, or a linear n-alkyl chain between 2 and 20 carbons in length;
each X⁻ is independently an anion selected from Cl⁻, Br⁻, I⁻, F⁻, SO₄²⁻, PO₄³⁻, CO₃²⁻, CH₃SO₃⁻, CF₃SO₃⁻, BF₄⁻, TsO⁻, AcO⁻, BzO⁻ and NTf₂⁻.

4. The polyionic silanization reagent of claim 3 of the formula V: 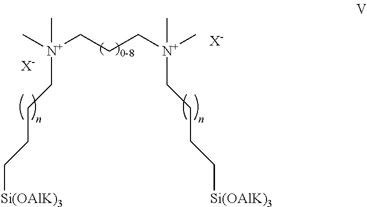
wherein:
Alk is methyl, ethyl, n-Pr, i-Pr, n-Bu, sec-Bu or t-Bu (or H after hydrolysis);
n is 0-7;
each X⁻ is an anion independently selected from Cl⁺, Br⁺, I⁻, F⁻, SO₄²⁻, CO₃²⁻, PO₄³⁻, CH₃SO₃⁻, CF₃SO₃⁻, BF₄⁻, TsO⁻, AcO⁻, BzO⁻ and NTf₂⁻.

5. The polyionic silanization reagent of claim 3 of the formulae VI and VII: 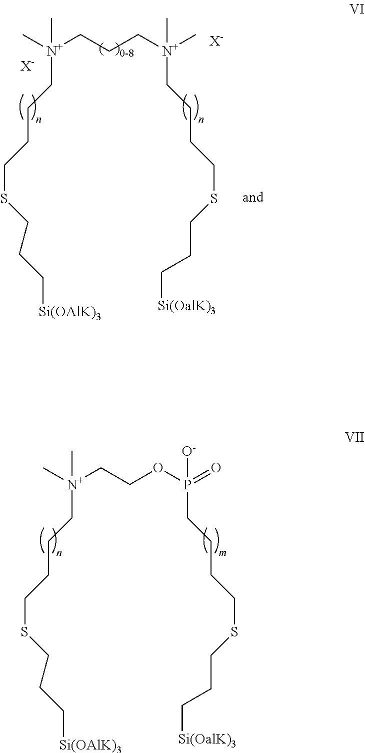
wherein:
Alk is methyl, ethyl, n-Pr, i-Pr, n-Bu, sec-Bu or t-Bu (or H after hydrolysis);
n is 0-7, m is 1-8; and
X⁻ is an anion selected from Cl⁻, Br⁻, I⁻, F⁻, SO₄²⁻, CO₃²⁻, PO₄³⁻, CH₃SO₃⁻, CF₃SO₃⁻, BF₄⁻, TsO⁻, AcO⁻, BzO⁻ and NTf₂⁻.

6. The polyionic silanization reagents of claim 3 wherein:
Alk is Me, Et, n-Pr, i-Pr, n-Bu, sec-Bu or t-Bu (or H after hydrolysis);
j is 1, k is 1, SP1 is 0, L2 is 0; and
EG is —[(CH₂CH₂O—)₁₋₃₀]-Me, or —[(CH₂CH₂O)₁₋₃₀]—H.

7. The polyionic silanization reagent of claim 3 wherein:
j is 1, k is 1, and Alk is either Me or Et

8. The polyionic silanization reagent of claim 3 wherein:
j is 2, k is 0, and Alk is either Me or Et

9. A method of coating a surface to prepare the polyionic surface coatings of claim 1 or 2, the method comprising:
a) obtaining and optionally cleaning a surface to be coated by application of soaps, bases, acids, solvents, water and/or alcohols, with or without optional scrubbing, or sonication;
b) optionally further rinsing the surface with water, alcohols, solvents, and then optionally drying the surface;
c) optionally hydroxylating the surface by either application of plasma cleaning technique, or exposing the surface to acidic solutions of peroxide or other oxidizing agents for a period of time, and then excess acids and oxidants and by-products are rinsed away before optional drying the surface;
i) wherein if the surface to be coated is a silicone or PDMS, the surface is hydroxylated before continuing;
d) treating the surface to be coated with the appropriate silanization agent in the appropriate solvent to render surface coated with a self assembled, reactive layer of the general formula: 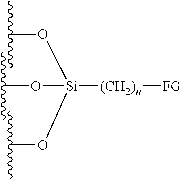
wherein:
the silyloxy group —(O)₃Si— is covalently bound to the surface;
n is 2-10,
and FG is a reactive functional group selected from: 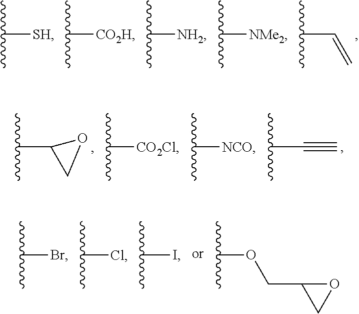
to form a reactive layer;
e) the reactive layer is reacted with a polyionic coupling agent containing an appropriate and complementary reactive functionality to that on the surface to achieve immobilization of the polyionic moiety upon the surface;
provided that:
i) if the FG is a thiol, then it is reacted with a polyionic coupling agent via a thiol-ene reaction, the complementary reactive functionality being either a terminal alkene or an alkyne;
ii) if the FG is a carboxylic acid or acid-chloride functionality, it is coupled by an established amide bond-forming procedure, with an amino functionalized polyionic coupling agent, the complementary reactive functionality that is a 1° or 2° amine;
iii) if the FG is a 1° amine, it is coupled with a polyionic isocyanate, a polyionic epoxide or by an established amide bond-forming procedure, with a carboxyl functionalized polyionic coupling agent, the reactive functionality of which is a carboxylic acid, acid chloride or activated ester;
iv) if the FG contains an epoxide or glycidyl moiety, then it is coupled with an amino functionalized polyionic coupling agent;
v) if the FG comprises a 3° dimethylamine it is quaternized with a polyionic chloride, polyionic bromide, polyionic iodide, or polyionic 1,3,2-dioxaphospholane 2-oxide,
vi) if the FG comprises a terminal alkene or alkyne, then it is reacted with a thiol-functionalized polyionic coupling agent via a thiol-ene reaction; and
vii) if the FG comprises an isocyanate, then it is reacted with a polyionic 1° amine, polyionic 2° monomethylamine or 10 polyionic alcohol.

10. The method of claim 9 comprising a reagent of claim 3 to coat a surface wherein:
a) the trialkoxysilyl group of any of the reagent of claim 3 undergoes a silanization reaction with the surface, immobilizing the polyionic compound upon a desired surface; and
b) the surface is optionally hydroxylated by application of plasma cleaning techniques, acidic peroxide or other oxidizing agents and optionally washed and dried prior to silanization.

11. (canceled)

12. The surface coating of claim 1 or 2, where the surface coating is present on materials that comprises a medical or dental device.

13. The surface coating of claim 12 whereby the surfaces are silicone, or PDMS, polyethylene, PET, PETG, PVC, polycarbonate (PC), PU, PMMA or their mixtures and copolymers.

14. A method of use of the surface coating of claim 13 whereby the surfaces comprise part of an indwelling medical device including catheters, endotracheal tubes, and shunts.

15. The surface coating of any one of claim 1 wherein the surface is a polymer including silicone, PDMS, polyethylene, PET, PETG, PVC, polycarbonate (PC), PU, PMMA, or their mixtures and copolymers.

16. The surface coating of claim 1 or 2 wherein the surface is any mineral and metal oxides including mica, silica, SiO₂, glass, calcium oxide, enamel, bone, steel, tooth enamel, tooth dentin, hydroxyapatite, kaolin, zirconia, aluminum, copper, chrome, chrome-cobalt, titanium, zinc, tin, and indium-tin.

17. The surface coating of claim 16 wherein the surface is present in a dental appliance and/or in the dental cavity such clear aligners, crowns, and implants.

18. The method of claim 10 whereby the coating reduces the incidence or rate of biofouling relative to the uncoated surface.
